(19) United States
(12) Patent Application Publication
(10) Pub. No.: US 2017/0224540 A1
(43) Pub. Date: Aug. 10, 2017

(54) BIODEGRADABLE, BIOBASED DIAPER

(71) Applicant: LMNF Inc., Farmingdale, NY (US)

(72) Inventors: Clive Li, Brooklyn, NY (US); Gary Metzger, Centerport, NY (US); Lauren Nichols, Saint James, NY (US); Lawrence Frisina, Smithtown, NY (US)

(73) Assignee: LMNF Inc., Farmingdale, NY (US)

(21) Appl. No.: 15/502,278

(22) PCT Filed: Aug. 8, 2015
(86) PCT No.: PCT/US15/44390

§ 371 Date: Feb. 7, 2017

Related U.S. Application Data
(60) Provisional application No. 62/034,922, filed on Aug. 8, 2014.

(51) Int. Cl. A61F 13/15; A61L 15/62; A61L 15/24; A61L 15/28; A61L 15/26; A61F 13/49; A61L 15/60

(52) U.S. Cl. CPC A61F 13/15252; A61F 13/49011; A61L 15/62; A61L 15/60; A61L 15/28; A61L 15/26; A61L 15/24

(57) ABSTRACT

The present invention relates to a disposable diaper that is biobased and/or biodegradable. The diaper is either made of wholly or partially renewable resources, at the same time can decompose in a compost site, and biodegrade in a conventional landfill as well as in marine conditions such as fresh, brackish or salt water.

### CLAIM OF PRIORITY

The present application claims the benefit of priority of U.S. Provisional Application Ser. No. 62/034,922, filed Aug. 8, 2014, which is incorporated herein by reference in its entirety.

### BACKGROUND OF THE INVENTION

For many years cloth diapers were used over and over again, being washed between each use. In the 1960's, disposable diapers were introduced to the market. Disposable diapers generally consisted of a liquid impervious plastic back sheet, an absorbent pat, and a liquid permeable plastic top sheet. Such disposable diapers were designed to be discarded after a single use.

Unfortunately, disposal of such diaper after used become a problem. A baby may use 6-8 diapers a day, leading up to more than 2000 diapers per year. Such disposable diapers cannot be recycled successfully because of the large amount of cellulose and other materials presented in the diaper. Disposal of such diapers in landfills contributes to the accumulation of garbage. The plastic materials do not break down under landfill conditions and remain in their original form for hundreds of years.

Attempts are made to alleviate this problem by using plastics which are “biodegradable”. U.S. Pat. No. 5,185,009 described using a biodegradable polyethylene. Polyethylene can be biodegradable in landfill, where there is no UV exposure only if it is copolymerized with a “truly biodegradable” polymer such as starch. The “truly degradable” part will be consumed by microbes and leaving behind polyethylene oligomers, which will take very long time for microbes to digest. Pat No. WO2009151439 A1 describes a method of making Polylactic Acid (PLA) fibers which can be further used in diaper applications. PLA is only compostable. It cannot decompose under landfill environment. Pat. No. EP0569154A1 and PE0569154B1 describe the use of aliphatic polyesters for the leak proof backing sheet and the water permeable sheet in a disposable diaper. Some of the aliphatic polyesters are biodegradable and can decompose in landfill.

A more pressing problem than the space taken in landfills, however, is the limited amount of resources to make diapers. Diapers made of fossil petroleum derived plastics is a potential problem. Petroleum is a limited resource that will only last for a few more decades. By using a biobased plastic can solve this problem. Unlike the petroleum derived plastics, biobased plastics are derived from renewable resources. These resources are predominantly plants such as trees, corns and sugar canes.

Pat. No. WO 2009/012284A1 describes a disposable diaper with back sheet and top sheet are made of a bi-component spun bond nonwoven material comprising a renewable polymer and a nonrenewable thermoplastic polymer. In the description, it was not mentioned to use a biodegradable material. In fact, it was claimed that the thermoplastic polymer is polypropylene, a material which is not derived from a renewable source and not biodegradable.

It is our view that a preferred disposable diaper should be biodegradable and are made of biobased or renewable materials.

The Biobased content of a diaper can be validated. The carbon in a biobased or renewable material is called Modern Carbon. Modern Carbon contains both Carbon-14 and Carbon-12. The carbon in a petroleum based materials is call Fossil Carbon. The Carbon-14 in the Fossil Carbon have undergone radioactive decay and leaving only carbon-12. ASTM D6866 is a standard method to determine the biobased or renewable content of an organic solid, liquid or gas. It is done by deriving a ratio of the amount of carbon-14 in a specimen to that of a reference standard. Percentage of Carbon-14 is reported. A zero percent (0%) Carbon 14 indicates a fossil carbon source. A hundred percent (100%) Carbon-14, likewise indicates an entirely Modern Carbon resource.

### SUMMARY OF THE INVENTION

The present invention relates to a disposable diaper that is biobased and biodegradable. In this preferred embodiment, the disposable diaper comprises an outer sheet of biobased and/or biodegradable thermoplastic which is impermeable to aqueous media; an inner sheet of biobased and/or biodegradable thermoplastic which is permeable to aqueous media; an absorbent pad made of natural fibers and a biobased superabsorbent; biobased and/or biodegradable hook and loop fastening tabs and fastening mat attached to the outer layer; a biobased and/or biodegradable elastic waist strip attached to the outer layer, and biobased and/or biodegradable side sheets which are impermeable to aqueous media so as to avoid fluid leakage.

A biobased material is a material constructed from renewable natural sources, such as corn, trees and/or plants, and/or animals such as mammals and/or birds such as domestic mammals and domestic birds. A biobased material is a carbon based material that is not derived from a petroleum source. Biobased materials also include sand, silica and inorganic salts such as calcium or sodium carbonate and calcium or sodium bicarbonate. These materials may optionally be incorporated into the carbon based, biobased material

The biobased, biodegradable thermoplastic, the absorbent pad, the biodegradable tabs and the biodegradable elastic are all constructed of materials that primarily, substantially, essentially or completely will decompose in composting and/or land fill and/or marine conditions. Bacterial decomposition is involved in all of these conditions. However, composting and marine decomposition also involve elevated temperature and non-bacterial hydrolysis.

The non-bacterial hydrolysis of the components of the diaper of the invention can be enhanced by incorporation of carbonate and/or bicarbonate microparticles within these components. The presence of a base and moisture accelerates hydrolysis of these diaper components.

The thermoplastic polymers including polyesters, polyolefins, cellulosic polymers and/or combinations thereof are polymers having the typical and usual thermoplastic property described in the Definitions section. The synthetic polyesters may be formed either from a combination of a diol and diacid or from an hydroxy acid. Additionally, some polyesters may be formed enzymatically and/or by bacterial or fungal production from feedstocks such as fatty acids. Combinations of these kinds of polymers also may constitute the thermoplastic polymer.

The absorbent pad is constructed of biobased and/or biodegradable superabsorbent with biodegradable natural fibers made from cellulose, mammal hair or avian feathers and may be chemically modified by addition of biodegradable linkage moieties and/or side chain moieties.

The tabs and elastic components are also constructed of biodegradable compositions as described below.

### BRIEF DESCRIPTION OF THE DRAWING

A more complete understanding of the invention will be apparent from the Detailed Description taken in conjunction with the accompanying drawings, in which:

FIG. 1 is an exploded perspective view showing the components of a diaper according to the invention

### DETAILED DESCRIPTION OF THE INVENTION

### Definitions

The term biobased means a material, composition and/or substance that is derived, produced or synthesized in part or in whole from a renewable source such as a plant or animal source. For example polylactic acid (PLA) which is produced by chemical and/or enzymatic conversion of corn starch is a biobased polyester. Similarly polyhydroxyalkanotes such as polyhydroxybutyric acid (PUB) are polyesters produced by bacterial or enzymatic conversion of carbohydrates such as sugar or glucose or cannola oil.

The term compostable means that a carbon based material can be bacterially, enzymatically and/or hydrolytically degraded under conditions typically present in organic composting processes. Such conditions usually involve warm temperatures, a combination of aerobic and anerobic bacterial activity and non-bacterial hydrolysis. Typically, a compostable material will be decomposed by bacterial and/or enzymatic action and/or by hydrolysis to a point where the compostable material is no longer recognizable or distinguishable from humus, however, the compostable material typically is not converted at this point to carbon dioxide and water plus inorganic residue.

The term biodegradable means that a carbon based material can be bacterially and/or enzymatically decomposed under conditions that are typically present in landfills. Such conditions usually include deep burial so that anaerobic bacterial and/or enzymatic activity typically will be the primary mode of decomposition. Such conditions typically do not include substantial non-bacterial hydrolysis but hydrolysis may occur to some extent. Typically, the biodegradable material is converted primarily, substantially, essentially or completely to ultimate decomposition products such as carbon dioxide, water and inorganic residue. The time needed for such full decomposition is usually substantial but bacterial and/or enzymatic decomposition will begin shortly after burial.

The term biodegradable also includes marine biodegradation. Submersion of a carbon based material in fresh, brackish or salt water enables bacterial and hydrolytic decomposition such that marine biodegradation occurs. Ultimately the decomposition produces final decomposition materials such as carbon dioxide, water and inorganic residue.

The term hook and loop means a device that attaches two parts together by connecting the hook and loop together. The hook and loop may be a single hook and loop or may be a series of hooks and loops, especially mini hooks and loops such as are present in “velcro” strips.

The term liquid impermeable means that a substance formed as a film or sheet will not allow water and/or an aqueous medium to pass through it. Such a substance may exhibit leakage such that a miniscule amount of water or aqueous medium may eventually make its way through the substance over a significant period of time such as 2 to 6 hours or longer. Preferably, miniscule leakage is also not present. Such a substance may allow air to pass through but air permeability is not a requirement.

The term liquid permeable means that a substance formed as a film or sheet will allow water and/or and aqueous medium to pass through it. The permeability may be the result of pores or may be the result of a non-porous but water diffusible material. Water diffusion through such a permeable material is typically passive.

The term superabsorbent means that a substance that absorbs aqueous medium is able to take on a significant weight proportion of aqueous medium relative to its dry weight. Preferably, such a substance has a dry feel even though it has absorbed a significant weight portion of aqueous medium

The term thermoplastic means that a polymer is solid at ambient temperature, is molten, plastic, flowable and/or deformable at elevated temperature and reverts to its prior condition upon cooling from the elevated temperature to ambient temperature.

The term elastic means that a substance can be stretched and/or deformed under stress and will return to its original shape, size and consistency upon discontinuation of the stress.

The term polyester means a polymer in which monomer units are linked together by ester (—COO—) groups, usually formed by polymeric esterification of a diol and a diacid or esterification of an hydroxy acid. Examples of the hydroxy acid polyester include polylactic acid (PLA), polyglycolic acid (PLG), polylactic/glycolic acid (PLG), polyhydroxybutyric acid (PHB), polyhydroxypropanoic acid. The polyester may also or alternatively be an aliphatic polyester, an aromatic polyester of a combination thereof. Examples of the diacid polyesters include polybutylenesuccinate (PBS) and polyhexylene adipate (PHA). Aromatic polyesters are typically and usually formed by condensation of aliphatic diols and aromatic dicarboxylic acids. Typical production techniques include terphthalate substitution for the diacid or inclusion of terphthalate along with the aliphatic diacid. Examples include polybutylene adipate terephthalate (PBAT), polybutylenesuccinate terphthalate, polyhexylene succinate terphthalate, polybutylene terphthalate, polyhexylene terphthalate, polybutyleneadipate terphthalate, polyhexylene adipate terphthalate.

The term polyolefin means a polymer formed from an organic compound having at least one carbon-carbon double bond that is not aromatic, with an empirical formula CₙH₂ₙ. Examples include polyethylene, polypropylene, polybutylene and polyisobutylene. A polyolefin may be biobased or petroleum based. Rubber is a typical generic name for vulcanized polyisobutylene, which is polyisobutylene cross-linked with sulfur.

The term petroleum based polyolefin means a polyolefin that is produced from a petroleum feedstock.

The term aqueous medium in the context of the invention means human or mammal urine, feces and similar human or mammal excrement.

### DESCRIPTION OF EMBODIMENTS

The disposable diaper is constructed primarily, substantially or essentially of biobased and/or biodegradable materials so that the disposable diaper will decompose in a moderate amount of time in a landfill, in a burial site, in a compost site and/or in a garbage dump. Typically, the decomposition of the disposable diaper produces humus and residual material that is not recognizable as diaper material. The humus and residual material are of a chemical nature that enables them to be assimilated by resident organisms present in soil, landfills, compost sites and dumps. Preferably, the decomposition results primarily, substantially or essentially in further reduction of the diaper materials into carbon dioxide, water and inorganic residue.

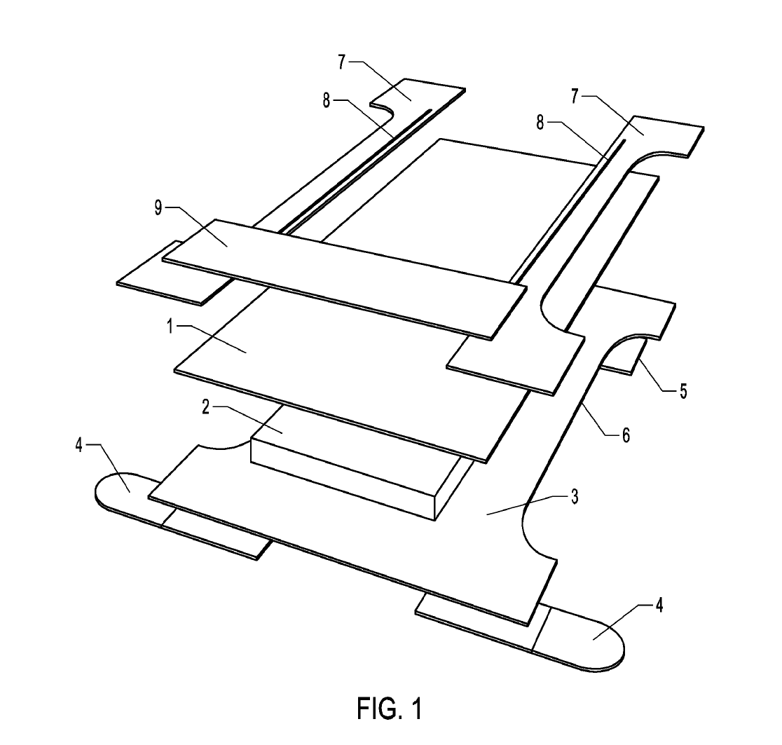

FIG. 1 illustrates each component of a disposable diaper. While FIG. 1 illustrates an embodiment of the disposable diaper according to the invention, other configurations of a disposable diaper constructed primarily, substantially or essentially of biobased material with the option of inclusion of some petroleum based material will be clearly apparent to the skilled practitioner.

As illustrated in FIG. 1, the inner sheet 1 is aqueous permeable which allows aqueous liquid and other excrements to pass through to an absorbent pad 2 underneath. The inner sheet 1 is made of biobased and/or biodegradable material. The preferred biobased content is greater than 25%.

The outer sheet 3 is aqueous impermeable. It is made of biobased and/or biodegradable material. The biobased content is preferred to be greater than 25%.

The absorbent pad 2 is a blend of natural fibers and a biobased and/or biodegradable superabsorbent. The natural fiber can be cellulosic such as cellulose fiber or bamboo fiber. The natural fiber can also be protein based such as animal hair or poultry feather. The preferred biobased content for the fiber material in the absorbent pad 2 is greater than 90%. Superabsorbent can be cellulosic based, starch based or protein based. Starch based supper absorbent is a copolymer or a grafted polymer containing starch or polysaccharides. Protein based super absorbent is made from protein such as keratin from feathers. The preferred biobased content for the superabsorbent in the absorbent pad 2 is greater than 25%.

The illustrated embodiment includes fastening tabs 4 at two corners. The tabs 4 can be attached to the fastening mat 5 on other end of the diaper by a hook and loop type connection to secure the diaper in position on an infant. To ensure the fastening device to hold firm, the flexural modulus of the material of fastening tabs 4 should be greater than 60000 psi by ASTM D790 testing method. Both the fastening tabs 4 and the fastening mat 5 are made of biobased and/or biodegradable material. The preferred biobased contents are greater than 25%.

The illustration in FIG. 1. showing that the diaper has a narrow section 6 which fits the leg of an infant. It is desirable to include elastic side sheets 7 to avoid leakage. The side sheets are aqueous impermeable. The preferred biobased content of the side sheets are greater than 25% Attached to side sheets 7 are biobased and/or biodegradable rubber bands 8, making the sides sheets 7 elastic.

For a diaper to hold comfortably tight on a body such as that of an infant, a biobased and/or biodegradable elastic waist strip 9 is attached to the diaper. The preferred biobased content of the elastic waist strip 9 is greater than 25%.

The inner sheet 1, outer sheet 3, and the side sheets 7 are made of biodegradable aliphatic polyester materials derived from biobased and/or renewable sources. Biobased polyhydroxyalkanoates and biobased polybutylene succinate are examples of such biodegradable material. The sheets can be formed by spun bond method, thermal bond method, stitch bond method, needle punch method, or a melt blown method. These sheets can also be coated with a layer of cellulose fiber such as paper to improve the mechanical property.

### Materials for Diaper Components

The permeable inner sheet may be composed of a biobased or mixed biobased and petroleum based biodegradable thermoplastic polymer such as a polyester, chemically modified cellulosic polymer, polyamide and/or a polyolefin such as polypropylene is appropriate. The sheet may be extrusion or cast formed from such thermoplastic polymer pellets. If the sheet is to be porous, the pores may be formed in a conventional, known manner such as by air jet formation or laser formation during extrusion or casting or by passing the sheet through a roller mill wherein the rollers have microspikes suitable for forming micropores on the sheet. Additionally, a porous sheet can be formed by incorporating microparticles of a water soluble substance such as sodium chloride and passing the sheet through a water bath after its formation. The water bath dissolves the microparticles so as to produce pores.

If the sheet is to be non-porous but water permeable, a thermoplastic polymer such as a polyester, chemically modified cellulosic polymer, polyamide and/or a polyolefin such as polypropylene is appropriate. Water loving factors such as hydrophilic groups, crystallinity, hydrophilic side chains and similar polymeric characteristics enable water to pass through the polymer by diffusion and/or hydrostatic interaction. In addition, water transmission can be increased in such polymers by incorporating cellulosic micro fibers such as cotton, wood, paper, bamboo microfibers into the polymer film. Preferred permeable polymers include polylactic acid, cellulose acetate, polyhydroxybutyric acid and combinations thereof.

Mixtures of water permeable polymers may also be used to form the permeable inner sheet. Up to about 20% by weight of cellulosic microfibers may be incorporated into the sheet. Combinations of polyester, cellulosic polymers and/or polyolefins ranging from essentially all polyester to approximately a majority of polyester with the remainder being one or more cellulosic and/or olefinic polymers may be used. Preferably, the weight percentage for this mixture ranges from about 100% polyester to about 60% polyester with the remainder being cellulosic and/or olefinic polymers, more preferably from about 100% to about 75% polyester with the remainder being cellulosic and/or olefinic polymers and most preferably about 100% polyester to 85% polyester with the remainder being cellulosic and/or olefinic polymers. The polyester and cellulosic and/or olefinic polymers in this construction are biobased and biodegradable.

The impermeable outer sheet may also be composed of biobased or mixed biobased and petroleum based polymers. Polymers such as polyesters, polyamides, polyolefins and specifically, polyhydroxybutyric acid, polybutylenesuccinate, polyhexylene adipate, polypropylene and similar polymers may be formed into the impermeable outer sheet. Forming the outer sheet by extrusion or casting and adjusting the parameters so that the sheet structure is substantially amorphous. Incorporation of hydrophobic groups, hydrophobic side chains and cross-linking the polymers enhances the impermeability of the outer sheet.

While the outer sheet may be essentially all biobased and/or biodegradable polyester, the outer sheet may also incorporate petroleum based polyolefin as well. The weight percent range of biobased biodegradable polyester for the outer sheet may be from essentially all polyester to a majority or slightly less than a majority percentage. Preferably the biobased, biodegradable polyester may range from about 100% to slightly less than 50% by weight with the remainder being a petroleum based polyester or polyolefin. More preferably, the biobased, biodegradable polyester may range from about 100% to about 60% by weight with the remainder being a petroleum based polyester and/or polyolefin. Most preferably, the biobased, biodegradable polyester may range from about 100% to about 75% by weight with the remainder being a petroleum based polyester and/or polyolefin. In all instances, the petroleum based polyester preferably may also be biodegradable.

The pad is formed of at least two components: an absorbent matrix and a superabsorbent filler particles. Cellulosic materials, mammal hair and/or avian feathers are useful as the matrix. Cotton fibers , wood fiber, wood fibers treated to remove lignin, bamboo fiber, bovine hair, pullum feathers are examples of material sources for the matrix. These materials can be chemically and/or mechanically treated to expand and micronize the fibers thereby increasing their surface area. For example, chemical conversion of these fiber materials into hydrogels by cross-linking enables superabsorbent properties. Examples include cellulosic microfibers chemically treated to modify cellulosic hydroxyl and carboxyl groups and form ester, ether and similar cellulosic groups such as carboxymethyl cellulose, ethyl cellulose and similar cellulosic materials. Superabsorbent filler can absorb aqueous media and aqueous liquid more than 100 times its dry weight. The biobased and/or biodegradable superabsorbent ca be composed of starch copolymers, such as starch-sodium polyacrylate copolymers. The preferred aqueous liquid absorbency of the superadsorbent filler is more than 130 times its dry weight

### Embodiments and Examples

Embodiments of the biobased and/or biodegradable disposable diaper of the invention may be designed and constructed to include the following features:
a) A biobased and/or biodegradable outer sheet impermeable to aqueous liquid;
b) A biobased and/or biodegradable inner sheet permeable to aqueous liquid and other body excrements;
c) An absorbent pad made of natural fibers and biobased and/or biodegradable superabsorbents;
d) Biobased and/or biodegradable side sheets to avoid fluid leakage;
e) Biobased and/or biodegradable fastening tabs and fastening mat to secure the diaper in position on a body;
f) A biobased and/or biodegradable elastic waist strip to comfortably secure the diaper in position on a body.

These embodiments of the biobased and/or biodegradable disposable diapers of the present invention preferably include an outer sheet made of aliphatic polyester. Preferably the aliphatic polyester is polyhydroxyalkanonate (PHA). Preferably, the PHA is biobased has a biobased content greater than 25%. Preferably, the PHA is biobased and has greater than 25% modern carbon.

The biobased and/or biodegradable disposable diapers of these embodiments may alternatively or additionally include an outer sheet of an aliphatic polyester which is polybutylene succinate (PBS) or its copolymer such as polybutylene succinate adipate (PBSA). Preferably, the PBS or PBSA is biobased and has a biobased content greater than 25%. Preferably, the PBS or PBSA is biobased and has greater than 25% modern carbon.

The biobased and/or biodegradable disposable diapers of the foregoing embodiments may alternatively or additionally include an impermeable outer sheet made of biodegradable aromatic polyester. Preferably, the biodegradable aromatic polyester is polybutylene succinate terephthalate (PBAT). Alternatively, the outer sheet is made of cellulosic fiber treated with a biodegradable polymer. As a second alternative, the outer sheet is made of a biodegradable polymer thin film laminated on a biodegradable nonwoven material. Preferably, the biodegradable nonwoven is made of polylactic acid derived from a plant source, preferably corn.

Alternatively, the embodiments of the biobased and/or biodegradable disposable diapers of present invention include an outer sheet is made of a combination of aliphatic polyester and aromatic polyester. Preferably, the combination is biobased and the biobased content is greater than 25%. Preferably, the combination is biobased and has greater than 25% modern carbon.

These embodiments of the biobased and/or biodegradable disposable diapers of the present invention preferably include an inner sheet made of aliphatic polyester. Preferably the aliphatic polyester is PHA. Preferably, the biobased content greater than 25%. Preferably, the PHA is biobased and has greater than 25% modern carbon. Alternatively, the inner sheet is made of an aliphatic polyester that is PBS or its copolymer such as PBSA. Preferably, the PBS or PBSA is biobased and has a biobased content greater than 25%. Preferably, the PBS or PBSA is biobased and has greater than 25% modern carbon. Alternatively, the inner sheet can be made of aromatic polyesters. Preferably, the aromatic polyester is PBAT. As an additional alternative, the foregoing polyesters for the inner sheet may also be partially made of cellulosic fiber. Preferably, the cellulosic fiber is bamboo fiber. Alternatively, the cellulosic fiber is cotton fiber. An example of the inner sheet is one made of polylactic acid and bamboo fiber. Another example of the inner sheet is one made of polylactic acid and cotton fiber. A further example of an inner sheet is one made of polylactic acid nonwoven material, e.g., a felted polylactic acid thread, fiber or material.

Alternatively, the embodiments of the biobased and/or biodegradable disposable diapers of the present invention include an inner sheet is made of a combination of aliphatic polyester and aromatic polyester. Preferably, the combination is biobased and the biobased content is greater than 25%. Preferably, the combination is biobased and has greater than 25% modern carbon.

These embodiments of the biobased and/or biodegradable disposable diapers of the present invention include side sheets are made of aliphatic polyester. Preferably, the side sheets are made of an aliphatic polyester that is PHA. Preferably, the PHA is biobased and the biobased content is greater than 25%. Preferably, the PHA is biobased and has greater than 25% modern carbon. Alternatively, the side sheets are made of an aliphatic polyester that is PBS or its copolymers PBSA. Preferably, the PBS or PBSA is biobased and the biobased content is greater than 25%. Preferably, the PBS or PBSA is biobased and has greater than 25% modern carbon. As another alternative, the side sheets are made of biodegradable aromatic polyester. Preferably, the biodegradable aromatic polyester is PBAT. As a further alternative, the side sheets are made of cellulosic fiber treated with a biodegradable polymer. As another alternative, the side sheets are made of a biodegradable polymer thin film laminated on a biodegradable nonwoven material. Preferably, the biodegradable nonwoven is made of polylactic acid.

As a further alternative, the embodiments of the biobased and biodegradable disposable diapers of the present invention include side sheets made of a combination of aliphatic polyester and aromatic polyester. Preferably, the combination is biobased and the biobased content greater than 25%. Preferably, the combination is biobased and has greater than 25% modern carbon.

The embodiments of the biobased and/or biodegradable disposable diapers of the present invention include fastening tabs and a fastening mat made of a combination of aliphatic polyester and aromatic polyester. Preferably, the combination is biobased and the biobased content is greater than 25%. Preferably, the combination is biobased and has greater than 25% modern carbon. As an alternative, the fastening tabs and the fastening mat are made of polypropylene. Preferably, the polypropylene is derived from plant material such as corn or soybeans. Preferably, the polypropylene is biobased and the biobased content is greater than 25%. Preferably, the polypropylene is biobased and has greater than 25% modern carbon. As an alternative, the fastening tabs and the fastening mat are made of polylactic acid.

The embodiments of the biobased and/or biodegradable disposable diapers of the present invention include an elastic waist strip is made of a combination of aliphatic polyester and aromatic polyester. Preferably, the combination is biobased and the biobased content is greater than 25%.

Preferably, the combination is biobased and has greater than 25% modern carbon. As an alternative, the elastic waist strip is made of a polylactic acid. As a further alternative, the elastic waist strip is made of a polypropylene. Preferably, the polypropylene is biobased and the biobased content is greater than 25%. Preferably, the polypropylene is biobased and has greater than 25% modern carbon.

The embodiments of the biobased and/or biodegradable disposable diapers of the present invention include an absorbent pad constructed of natural absorbent fibers. Preferably, the natural fiber is cellulose. Alternatively the natural fiber is bamboo fiber. Alternatively, the natural fiber is keratin hair. Alternatively, the natural fiber is keratin feather. Alternatively, the natural fiber is cotton fiber.

As an integral feature of the pad component of embodiments of the biobased and/or biodegradable disposable diapers of present invention, the superabsorbent fillers are starch based. Preferably, the superabsorbent has a biobased content greater than 25%. Preferably, the superabsorbent has greater than 25% modern carbon. Alternatively, the superabsorbent is protein based. Alternatively, the superabsorbent is keratin based.

The embodiments of the biobased and/or biodegradable disposable diapers of the present invention are produced by formation of the outer sheet, inner sheet, or the side sheets by a spun bond method. Alternatively, the outer sheet, inner sheet, or the side sheets are formed by a thermal bond method. Alternatively, the outer sheet, inner sheet, or the side sheets are formed by a stitch bond method. As a further processing feature, the inner sheet is formed by a needle punch method. As a further processing feature, the outer sheet is formed by a melt blown method.

In all of the foregoing embodiments, the combination of polyesters, polyolefins and cellulosic polymers as discussed in the foregoing Materials Section may be incorporated as the inner and outer sheets, side sheets, tabs, mat and elastic waist strip. In all of the foregoing embodiments, the cellulosic materials, mammal hair and/or avian feathers as discussed in the foregoing Materials Section may be incorporated as the several layers of the absorbent pad.

As a further feature for embodiments of the biobased and/or biodegradable diapers of the invention, the thermoplastic polymers may include inorganic components such as talc, mica, calcium carbonate, sodium bicarbonate and/or sodium carbonate. The basic inorganic components such as bicarbonates and carbonates may be incorporated as microparticles in the films and absorbent of these embodiments. These basic microparticles may also be coated with gelatin.

Inclusion of the basic microparticles facilitates hydrolytic decomposition of the thermoplastic polymers such as polyesters. Hydrolysis of the ester linkages in the presence of base and water is well-known. With such incorporation, contact of the diaper components with an aqueous medium triggers basic hydrolysis. In practical application, moisture from urine will begin this process. With such basic microparticles, degradation in landfills and in marine conditions and under anerobic conditions are accelerated and hydrolysis activity is heightened.

### Summary Statements

The inventions, examples, and results described and claimed herein have many attributes and embodiments include, but not limited to, those set forth or described or referenced in this application.

All patents, publications, scientific articles, web sites and other documents and references mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such paten, publication, scientific article, web site, electronically available information, text book or other referenced material or document.

The written description of this patent application includes all claims. All claims including all original claims are hereby incorporated by reference in their entirety into the written description portion of the specification and the right is reserved to physically incorporate into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims

The invention has been described broadly and generically herein. Each of the narrower species and subgeneric groupings falling within the generic disclosure also form part of the invention.

It is also to be understood that as used herein and in the appended claims, the singular forms “a,” “an,” and “the” include plural reference unless the context clearly dictates otherwise. Also, the term and/or includes both arrangements, for example, “X and/or Y” means “X” or “Y” and both of “X” and “Y”. The letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

1. A biobased biodegradable disposable diaper comprising:
a) A biobased and biodegradable outer sheet impermeable to aqueous medium;
b) A biobased and biodegradable inner sheet permeable to aqueous medium;
c) An absorbent pad made of natural fibers and biobased superabsorbents;
d) Biobased and biodegradable side sheets with attached biodegadable rubber bands so as to provide elasticity to the side sheets;
e) Biobased and biodegradable fastening tabs and fastening mat to secure the diaper in position on a body;
f) A biobased and biodegradable elastic waist strip to secure the diaper in position on a body; and
g) the outer sheet, inner sheet, side sheets, rubber bands, tabs, mat and strip are constructed at least of one or more biodegradable polymers selected from hydroxyalkanoic acid polyesters, polyol/diacid polyesters, cellulosic polymers or a combination thereof; the absorbent pad is a blend of natural fiber and biodegradable superabsorbent; and the biodegradable polymers and blend include microparticles of carbonate or bicarbonate salt.

2. (canceled)

3. A diaper according to claim 1 wherein the one or more biodegadable polymers include at least an aliphatic ester that is polybutylene succinate, polybutylene succinate adipate, polyhydroxybutyric acid, polylactic acid, polyglycolic acid, polylactic/glycolic acid, polyhexylene adipate, polyhexylene succinate, polybutylene adipate or any combination thereof.

4. A diaper according to claim 1 wherein the one or more biodegradable polymers include at least an aromatic polyester that is polybutylene succinate terphthalate, polyhexylene terphalate, polybutylene terphalate, polyhexylene adipate terphthalate, polybutylene adipate terphthalate or any combination thereof.

5. A diaper according to claim 1 wherein the one or more biodegradable polymers include at least a cellulosic polymer that is a natural or chemically modified cellulose material.

6. A diaper according to claim 1 wherein the inner sheet is made of polylactic acid and bamboo fiber or cotton fiber.

7. A diaper according to claim 1 wherein the inner sheet is made of polylactic acid nonwoven material.

8. A diaper according to claim 1 wherein the biodegradable polymer is biobased and its biobased content is greater than 25%.

9. A diaper according to claim 8 wherein the biodegradable polymer has greater than 25% modern carbon.

10. A diaper according to claim 1 wherein the pad is constructed of natural fibers selected from cellulosic fiber, protein fiber or keratin fiber.

11. A diaper according to claim 10 wherein the pad also includes a superabsorbent formed from modified starch, cellulosic, keratin or protein material functioning as hydrogel and/or micronized and/or expanded fibers.

12. A diaper according to claim 1 wherein the fastening tabs, fastening mat and elastic waist strip are made of a biobased and biodegradable polymer selected from the group consisting of polylactic acid, aliphatic polyester, aromatic polyester, and any combination thereof.

13. A biobased biodegradable disposable diaper according to claim 1 wherein the inner sheet is made of polylactic acid nonwoven fibers and bamboo nonwoven fibers laminated with cellulosic paper; the outer sheet and side sheets are made of polylactic acid nonwoven fibers laminated with polybutylene adipate terephthalate film; the absorbent pad is made of cellulosic fiber combined with a starch based superabsorbent particles; the hook and loop tabs, the rubber bands and mat are made of polypropylene; and the waist strip is made of a combination of polylactic acid and elastomer nonwoven fibers; and the polymers, cellulosic paper, cellulosic fiber, starch based superabsorbent contain distributed throughout basic miocroparticles of carbonate and/or bicarbonate salt.

14. A biobased biodegradable disposable diaper according to claim 1 wherein the inner sheet is made of polylactic acid nonwoven fibers laminated with cellulosic paper; the outer sheet and side sheets are made of polylactic acid nonwoven fibers laminated with polyhydroxyalkanoate film; the absorbent pad is made of cellulose fiber combined with starch based superabsorbent particles; the hook and loop tabs, rubber bands and mat are made of polylactic acid; the waist strip is made of a blend of polylactic acid and polyhydroxyalkanoate and elastomer; and the polylactic acid, polyhydroxyalkanoate, cellulose fiber, starch based superabsorbent particles contain distributed throughout basic microparticles of carbonate and/or bicarbonate salt.
